(19) United States
(12) Patent Application Publication
(10) Pub. No.: US 2016/0058334 A1
(43) Pub. Date: Mar. 3, 2016

(54) ACTIVITY STATUS INFORMATION COMPUTATION SYSTEM

(71) Applicant: Seiko Epson Corporation, Tokyo (JP)

(72) Inventor: Yusuke Takahashi, Matsumoto-shi (JP)

(21) Appl. No.: 14/820,537

(22) Filed: Aug. 6, 2015

(30) Foreign Application Priority Data
Aug. 27, 2014 (JP) 2014-172584

(51) Int. Cl. A61B 5/11; A61B 5/00

(52) U.S. Cl. CPC A61B 5/1118; A61B 5/72

(57) ABSTRACT

An activity status information computation system includes: a first information acquisition unit which acquires measured biometric information measured about a user; a second information acquisition unit which acquires personal biometric setting information set as personal information of the user; and a processing unit which carries out computation processing of activity status information of the user on the basis of the measured biometric information and the personal biometric setting information. when there is an alteration request about the personal biometric setting information, the processing unit carries out decision processing about a timing of reflecting the alteration of the personal biometric setting information on the computation processing of the activity status information, or decision processing about whether to reflect the alteration or not, according to an attribute of the personal biometric setting information.

This application claims priority to Japanese Patent Application No. 2014-172584, filed Aug. 27, 2014, the entirety of which is hereby incorporated by reference.

### BACKGROUND

1. Technical Field

The present invention relates to an activity status information computation system or the like.

2. Related Art

Pulse monitors and activity trackers execute processing for measuring activity status information such as calories burned and distance traveled, on the basis of personal biometric setting information such as the height, weight, age and gender of the wearer. For example, the calories burned are calculated on the basis of weight. As for the distance traveled, an estimate value of the stride length is calculated based on the height and the estimated value of the stride length is multiplied by the number of steps taken, thus calculating the distance traveled.

Recently, an activity status information computation system in which a pulse monitor or activity tracker and a smart device operate in cooperation with each other is invented. In such an activity status information computation system, the user can read activity status information on the smart device, and the pulse monitor or activity tracker can receive personal biometric setting information from the smart device and reflect an alteration of setting. An invention related to such an activity status information computation system is the related-art technique described in JP-A-2002-303545.

The pulse monitor or activity tracker is an apparatus which is constantly worn by the user and continues measuring measured biometric information. Therefore, there are cases where personal biometric setting information should be reflected on the apparatus during measurement.

However, there are cases where inconvenience occurs if personal biometric setting information received from the smart device is directly reflected on the apparatus. For example, the gender, date of birth and user ID usually do not change. If the gender, date of birth or user ID is changed, there is a high possibility that an erroneous input is made. Also, the values of the height and weight are unlikely to change greatly within a few hours. Since the calculation of the calories burned and distance traveled is closely related to personal biometric setting information, such an incorrect setting of personal biometric setting information causes a significant error in the result of measurement of activity status information.

Although it is conceivable that the smart device carries out check processing on the inputted or altered personal biometric setting information and shows a screen display to prompt the user to check the information, there may be cases where the user does not view or fails to view the display. Therefore, it is not necessarily effective.

### SUMMARY

An advantage of some aspects of the invention is to provide an activity status information computation system or the like which enables correct calculation of activity status information even if an erroneous instruction to alter personal biometric setting information is given.

An aspect of the invention relates to an activity status information computation system including: a first information acquisition unit which acquires measured biometric information measured about a user; a second information acquisition unit which acquires personal biometric setting information set as personal information of the user; and a processing unit which carries out computation processing of activity status information of the user on the basis of the measured biometric information and the personal biometric setting information. When there is an alteration request about the personal biometric setting information, the processing unit carries out decision processing about a timing of reflecting the alteration of the personal biometric setting information on the computation processing of the activity status information, or decision processing about whether to reflect the alteration or not, according to an attribute of the personal biometric setting information.

In the aspect of the invention, when there is an alteration request about the personal biometric setting information, decision processing about a timing of reflecting the alteration of the personal biometric setting information on the computation processing of the activity status information, or decision processing about whether to reflect the alteration or not, is carried out according to the attribute of the personal biometric setting information.

Thus, the activity status information can be correctly calculated even when an erroneous instruction to alter the personal biometric setting information is given.

In the aspect of the invention, when the attribute is a first attribute, the processing unit may carry out reflection processing of the alteration at a first timing corresponding to the alteration request, and When the attribute is a second attribute, the processing unit may not carry out the reflection processing or may carry out the reflection processing at a second timing that is later than the first timing.

With this configuration, it is possible to reflect an alteration of the personal biometric setting information at different timings according to the attribute, and not to reflect an alteration of the personal biometric setting information, depending on the attribute, or the like.

In the aspect of the invention, the processing unit may carry out the decision processing about the reflection timing or the decision processing about whether to reflect the alteration or not, on the basis of at least one of an amount of change in value of the attribute in the alteration request, time elapsed since the previous reflection processing of the personal biometric setting information, and time of use of a measuring apparatus for the personal biometric setting information, and on the basis of the attribute.

With this configuration, even in the case of an alteration of the personal biometric setting information with the same attribute, it is possible discriminate the timing of reflecting the alteration of the setting, and whether to carry out the reflection processing or not, or the like.

In the aspect of the invention, the attribute may be the weight of the user, and the processing may carry out the reflection processing of the alteration at a first timing corresponding to the alteration request when the time elapsed since the previous reflection processing is equal to or longer than a first time elapsed.

Thus, when a predetermined time or longer has passed since the previous reflection timing for the alteration of the setting, it is possible to immediately reflect the alteration of the weight setting on the activity status information even during measurement regardless of the amount of change in the weight, or the like.

In the aspect of the invention, the attribute may be the weight of the user, and the processing unit may decide not to carry out the reflection processing of the alteration when the time elapsed since the previous reflection processing is shorter than a second time elapsed which is shorter than the first time elapsed and the amount of change is equal to or greater than a predetermined amount of change.

With this configuration, it is possible not to reflect the alteration of the setting to greatly alter the weight on the computation processing of the activity status information within a short period following the previous reflection timing for the alteration of the setting, or the like.

In the aspect of the invention, the attribute may be the weight of the user, and the processing unit may carry out the reflection processing of the alteration at a second timing that is later than a first timing corresponding to the alteration request, when the time elapsed is equal to or longer than a second time elapsed and shorter than a first time elapsed and the amount of change is equal to or greater than a predetermined amount of change.

With this configuration, it is possible to put on hold the alteration of the setting to greatly alter the weight within a predetermined period following the previous reflection timing for the alteration of the setting, or the like.

In the aspect of the invention, the attribute may be the weight of the user, and the processing unit may carry out the reflection processing of the alteration at a first timing corresponding to the alteration request, when the time elapsed since the previous reflection processing is equal to or longer than a second time elapsed and shorter than a first time elapsed and the amount of change in the value of the attribute is less than a predetermined amount of change.

With this configuration, it is possible to immediately reflect the alteration of the setting to slightly alter the weight on the computation processing of the activity status information even during measurement, within a predetermined period following the previous reflection timing for the alteration of the setting, or the like.

In the aspect of the invention, the attribute may be the weight of the user, and the processing unit may carry out the reflection processing of the alteration at a second timing that is later than a first timing corresponding to the alteration request, when the time elapsed since the previous reflection processing is shorter than a second time elapsed which is shorter than a first time elapsed and the amount of change in the value of the attribute is less than a predetermined amount of change.

With this configuration, it is possible to put on hold the alteration of the setting to slightly alter the weight within a short period following the previous reflection timing for the alteration of the setting, or the like.

In the aspect of the invention, the attribute may be the height of the user, and the processing unit may carry out the decision processing about the reflection timing or the decision processing about whether to reflect the alteration or not, on the basis of the age or date of birth of the user.

With this configuration, it is possible to decide whether to reflect the alteration of the setting or not, or to decide the reflection timing when the alteration of the setting is to be reflected, on the basis of different criteria depending on the age of the user, or the like.

In the aspect of the invention, the attribute may be the height of the user, and the processing unit may carry out the reflection processing of the alteration at a first timing corresponding to the alteration request, when the age of the user is below a predetermined threshold, the time of use is shorter than a predetermined time of use, and the time elapsed is shorter than a predetermined time elapsed.

With this configuration, for example, when the user is under 18 years of age and the apparatus is in the initial stage of the start of use, and when a predetermined period has not passed since the timing when the previous alteration of the height setting is reflected, it is possible to immediately reflect the alteration of the height setting on the computation processing of the activity status information, or the like.

In the aspect of the invention, the attribute may be the height of the user, and the processing unit may carry out the reflection processing of the alteration at a second timing that is later than a first timing corresponding to the alteration request, when the age of the user is below a predetermined threshold, the time of use is equal to or longer than a predetermined time of use, and the time elapsed is shorter than a predetermined time elapsed.

With this configuration, for example, when the user is under 18 years of age and the apparatus is not in the initial stage of the start of use, and when a predetermined period or longer has not passed since the timing when the previous alteration of the height setting is reflected, it is possible to put on hold the alteration of the setting to alter the height, or the like.

In the aspect of the invention, the attribute may be the height of the user, and the processing unit may carry out the reflection processing of the alteration at a first timing corresponding to the alteration request, when the age of the user is below a predetermined threshold, the time of use is equal to or longer than a predetermined time of use, and the time elapsed is equal to or longer than a predetermined time elapsed.

With this configuration, for example, when the user is under 18 years of age and the apparatus is not in the initial stage of the start of use, and when a predetermined period has passed since the timing when the previous alteration of the height setting is reflected, it is possible to immediately reflect the alteration of the height setting on the computation processing of the activity status information, or the like.

In the aspect of the invention, the attribute may be the height of the user, and the processing unit may carry out the reflection processing of the alteration at a second timing that is later than a first timing corresponding to the alteration request, when the age of the user is equal to or above a predetermined threshold, the time of use is shorter than a predetermined time of use, and the time elapsed is shorter than a predetermined time elapsed.

With this configuration, for example, when the user is 18 years old or above and the apparatus is in the initial stage of the start of use, and when a predetermined period has not passed since the timing when the previous alteration of the setting is reflected, it is possible to put on hold the alteration of the setting to alter the height, or the like.

In the aspect of the invention, the attribute may be the height of the user, and the processing unit may decide not to carry out the reflection processing of the alteration when the age of the user is equal to or above a predetermined threshold, the time of use is equal to or longer than a predetermined time of use, and the time elapsed is shorter than a predetermined time elapsed.

With this configuration, for example, when the user is 18 years old or above and the apparatus is not in the initial stage of the start of use, and when only a short period has passed since the timing when the previous alteration of the height setting is reflected, it is possible to reject the alteration of the setting to alter the height, or the like.

In the aspect of the invention, the attribute may be the height of the user, and the processing unit may carry out the reflection processing of the alteration at a second timing that is later than a first timing corresponding to the alteration request, when the age of the user is equal to or above a predetermined threshold, the time of use is equal to or longer than a predetermined time of use, and the time elapsed is equal to or longer than a predetermined time elapsed.

With this configuration, for example, when the user is 18 years old or above and the apparatus is not in the initial stage of the start of use, and when a predetermined period or longer has passed since the timing when the previous alteration of the height setting is reflected, it is possible to put on hold the alteration of the setting to alter the height, or the like.

In the aspect of the invention, the attribute may be the date of birth or gender of the user, and the processing unit may carry out the reflection processing of the alteration at a second timing that is later than a first timing corresponding to the alteration request, when the time of use is equal to or longer than a predetermined time of use and the time elapsed is equal to or longer than a predetermined time elapsed.

With this configuration, when the apparatus is not in the initial stage of the start of use, and when a predetermined time has passed since the timing when the previous alteration of the setting of the date of birth or gender is reflected, it is possible to put on hold the alteration of the setting to alter the date of birth or gender, or the like.

In the aspect of the invention, the attribute may be the date of birth or gender of the user, and the processing unit may decide not to carry out the reflection processing of the alteration when the time of use is equal to or longer than a predetermined time of use and the time elapsed is shorter than a predetermined time elapsed.

With this configuration, when the apparatus is not in the initial stage of the start of use, and when a predetermined time has not passed since the timing when the previous alteration of the setting of the date of birth or gender is reflected, it is possible to reject the alteration of the setting to alter the date of birth or gender, or the like.

In the aspect of the invention, the attribute may be the date of birth or gender of the user, and the processing unit may carry out the reflection processing of the alteration at a second timing that is later than a first timing corresponding to the alteration request, when the time of use is shorter than a predetermined time of use and the time elapsed is shorter than a predetermined time elapsed.

With this configuration, when the apparatus is in the initial stage of the start of use and a predetermined time has not passed since the timing when the previous alteration of the setting of the date of birth or gender is reflected, it is possible to put on hold the alteration of the setting to alter the date of birth or gender, or the like.

### BRIEF DESCRIPTION OF THE DRAWINGS

The invention will be described with reference to the accompanying drawings, wherein like numbers reference like elements.

FIG. 1 shows an example of a system configuration according to an embodiment.

FIG. 2 is an explanatory view of an apparatus which realizes an activity status information computation system according to the embodiment.

FIG. 3 is a flowchart for explaining a flow of overall processing in the embodiment.

FIG. 4 is a flowchart for explaining a flow of classification processing.

FIG. 5 is a flowchart for explaining a flow of decision processing and reflection processing of an alteration of weight setting.

FIG. 6 is an explanatory view concerning the reflection of an alteration of weight setting.

FIGS. 7A to 7C are explanatory views of screen displays.

FIG. 8 is a flowchart for explaining a flow of decision processing and reflection processing of an alteration of height setting.

FIG. 9 is another flowchart for explaining a flow of decision processing and reflection processing of an alteration of height setting.

FIGS. 10A and 10B are explanatory views concerning the reflection of an alteration of height setting.

FIG. 11 is a flowchart for explaining a flow of decision processing and reflection processing of an alteration of date of birth or gender setting.

FIG. 12 is an explanatory view concerning an alteration of date of birth or gender setting.

FIGS. 13A and 13B show the external appearance of a measuring device according to the embodiment.

FIG. 14 shows the external appearance of the measuring device according to the embodiment.

FIG. 15 is an explanatory view about the installation of the measuring device and its communication with an information processing device.

### DESCRIPTION OF EXEMPLARY EMBODIMENTS

Hereinafter, an embodiment will be described. The embodiment described below is not to unduly limit the content of the invention described in the appended claims. Not all the configurations described in the embodiment are essential components of the invention.

### 1. Outline

A measuring device (pulse monitor, activity tracker or the like) of an activity status information computation system according to the embodiment is constantly worn by the user and continues measuring measured biometric information of the user. The activity status information computation system calculates activity status information of the user. To calculate the activity status information, personal biometric setting information of the user needs to be used.

Here, measured biometric information is information of the user that can be measured directly by a sensor or the like provided in the measuring device. The measured biometric information is, for example, pulse information measured by a pulse sensor, acceleration information measured by an acceleration sensor, and the like. The measured biometric information keeps changing according to the activity of the user.

The personal biometric setting information is personal information about the user. The personal biometric setting information is information mainly about the physical elements of the user, for example, height, weight, age, gender, maximum heart rate, resting heart rate, maximum oxygen uptake, resting oxygen uptake, body fat percentage, and also a set value indicating physical strength, a set value indicating physical condition, or the like. Information such as user ID is also included in the personal biometric setting information. The personal biometric setting information is information that hardly changes within a short period such as a second or minute. However, the personal biometric setting information may change over a long period such as a week or month.

The activity status information is information concerning the activity of the user specified on the basis of the measured biometric information and the personal biometric setting information. The activity status information is, for example, the calories burned or distance traveled by the user, number of steps taken, altitude change, velocity, exercise intensity, pulse threshold used for sleep determination, hours of sleep, quality of sleep, pulse zone setting, physical strength level, estimated oxygen debt (EPOC or excess post-exercise oxygen consumption), unique indicator value for the amount of activity calculated on the basis of these, or the like.

For example, the calories burned can be found using the following equation (1), where X represents calories burned (kcal), VO₂ represents oxygen uptake (ml/kg·min), T represents weight (kg), and H represents time (h).

X (kcal)=0.3×VO₂ (ml/kg·min)×T (kg)×H (h) (1)

As described above, the personal biometric setting information may change as measurement is continued over a long period. In order to calculate accurate activity status information at the time point, it is better to carry out computation processing based on the latest personal biometric setting information. To this end, the personal biometric setting information about which an alteration request is made needs to be reflected on the computation processing of the activity status information.

However, there are cases where inconvenience occurs when the personal biometric setting information received from the smart device is directly reflected on the computation processing of the activity status information.

For example, the gender, date of birth, and user ID usually do not change. When all of the gender, date of birth, and user ID are changed simultaneously, there is a possibility that another user is wearing the device anew. However, when one of the gender, date of birth, and user ID is changed, there is a high possibility that the information is erroneously inputted.

The values of the height and weight, too, are unlikely to change greatly within a few hours. However, when the user is underage, the height and weight can change greatly within a short period.

The calculation of the calories burned and distance traveled is closely related to the personal biometric setting information. Therefore, such an incorrect setting of the personal biometric setting information causes a significant error in the result of the measurement of the activity status information. However, not all alteration requests are erroneous.

Therefore, instead of immediately reflect an alteration of the personal biometric setting information without exception, it is necessary to determine whether an alteration request about the personal biometric setting information is correct or not, and to reflect the alteration of the personal biometric setting information on the computation processing of the activity status information only when the alteration request is correct.

Here, while it is conceivable that the smart device carries out check processing on the inputted or altered personal biometric setting information and shows a screen display to prompt the user to check the information, there may be cases where the user does not view or fails to view the display. Therefore, it is not necessarily effective.

Thus, in the activity status information computation system according to this embodiment, whether an alteration request about the personal biometric setting information is correct or not is automatically determined. This enables correct calculation of the activity status information even when an erroneous instruction to alter the personal biometric setting information is given.

### 2. Example of System Configuration

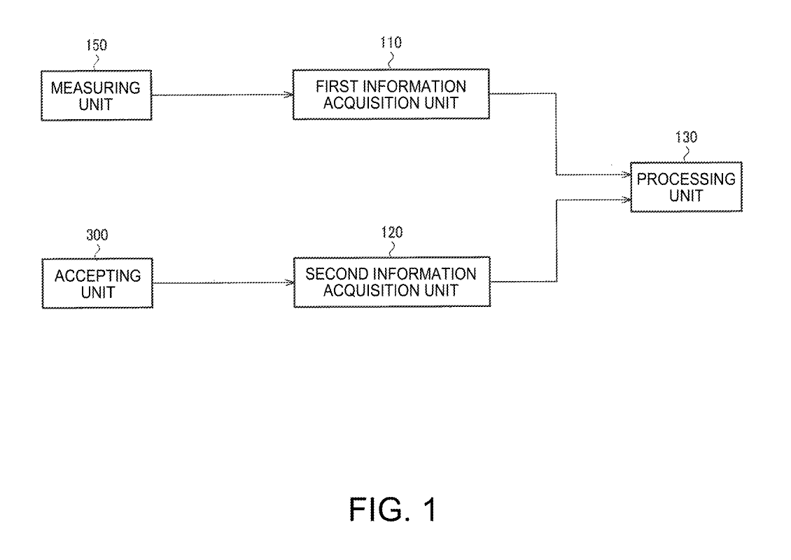

Next, an example of the configuration of an activity status information computation system 100 according to this embodiment is shown in FIG. 1.

The activity status information computation system 100 of this embodiment includes a first information acquisition unit 110, a second information acquisition unit 120, and a processing unit 130.

The first information acquisition unit 110 acquires measured biometric information measured about the user. The second information acquisition unit 120 acquires personal biometric setting information set as personal information of the user. The processing unit 130 carries out computation processing of activity status information of the user on the basis of the measured biometric information and the personal biometric setting information. The functions of the processing unit 130 can be realized by hardware such as various processors (CPU or the like) or ASIC (gate array or the like), or by a program or the like.

Also, when there is an alteration request about the personal biometric setting information, the processing unit 130 carries out decision processing about the timing of reflecting the alteration of the personal biometric setting information on the computation processing of the activity status information, or decision processing about whether to reflect the alteration or not, according to the attribute of the personal biometric setting information.

Here, the attribute of the personal biometric setting information refers to the type of the personal biometric setting information. The attribute of the personal biometric setting information refers to, for example, a type such as height, weight, age, gender, maximum heart rate, resting heart rate, maximum oxygen uptake, resting oxygen uptake, body fat percentage, and also a set value indicating physical strength or a set value indicating physical condition.

For example, when the attribute is a first attribute, the processing unit 130 carries out reflection processing of the alteration at a first timing corresponding to the alteration request. When the attribute is a second attribute, the processing unit 130 does not carry out the reflection processing or carries out the reflection processing at a second timing that is later than the first timing.

Here, the reflection processing refers to processing to reflect the personal biometric setting information about which an alteration request is made, on the computation processing of the activity status information carried out by the processing unit 130. Therefore, after the reflection processing is carried out, the processing unit 130 carries out the computation processing of the activity status information on the basis of the personal biometric setting information whose alteration is reflected.

The flow of the processing is described more specifically now. When the attribute of the personal biometric setting information about which an alteration request is made is, for example, weight (first attribute), the processing unit 130 reflects the personal biometric setting information immediately (at a first timing) even during measurement. When the attribute of the personal biometric setting information about which an alteration request is made is, for example, gender or user ID (second attribute), the processing unit 130 does not reflect personal biometric setting information. Alternatively, when the attribute of the personal biometric setting information about which an alteration request is made is, for example, height (second attribute), the processing unit 130 does not reflect the alteration of the personal biometric setting information during measurement, and instead carries out confirmation processing to the user at the point when the measurement is finished, and reflects the personal biometric setting information at the point when the alteration is confirmed as a normal alteration of the setting (at a second timing). The terms of first attribute and second attribute used in the description simply express two different attributes of the personal biometric setting information. Therefore, it is not the case that the first attribute is always weight while the second attribute is always gender, user ID, and height.

Thus, the alteration of the personal biometric setting information can be reflected at different timings according to the attribute. Depending on the attribute, it is possible not to reflect the alteration of the personal biometric setting information, or the like. That is, when an alteration request about the personal biometric setting information can be clearly determined as being due to an erroneous operation, the alteration of the personal biometric setting information is not reflected. On the other hand, when it can be determined that the alteration should be reflected immediately, the alteration of the setting is immediately reflected on the computation processing of the activity status information.

Thus, the activity status information can be correctly calculated even when an erroneous instruction to alter the personal biometric setting information is given.

The activity status information computation system 100 is not limited to the above configuration and various modifications can be made such as omitting a part of the components or adding another component. For example, the activity status information computation system 100 may include a measuring unit 150 and an accepting unit 300, as shown in FIG. 1.

The measuring unit 150 measures the measured biometric information of the user. The measuring unit 150 is, for example, a sensor unit, and more specifically, a pulse sensor, temperature sensor, and physical motion sensor or the like. The sensor unit is not necessarily formed by one sensor and may be formed by a plurality of types of sensors.

Here, the pulse sensor is a sensor for detecting a pulse sensor signal. For example, a photoelectric sensor or the like may be considered. However, a photoelectric sensor is not necessarily used as the pulse sensor, and other sensors such as a sensor using ultrasonic waves may also be used.

As the physical motion sensor, a motion sensor (acceleration sensor), pressure sensor (contact pressure sensor), position sensor (GPS receiver or the like), or the like is used. As the physical motion sensor, a plurality of types of sensors may be provided.

The accepting unit 300 accepts operation information by the user to alter the personal biometric setting information. The accepting unit 300 may be, for example, an operation input unit provided in a measuring apparatus main body, smart device (mobile terminal device, information processing device), or the like. The operation input unit is, for example, a button, switch, touch panel or the like. The accepting unit 300 may also be a communication unit which accepts the personal biometric setting information from an external device via a network.

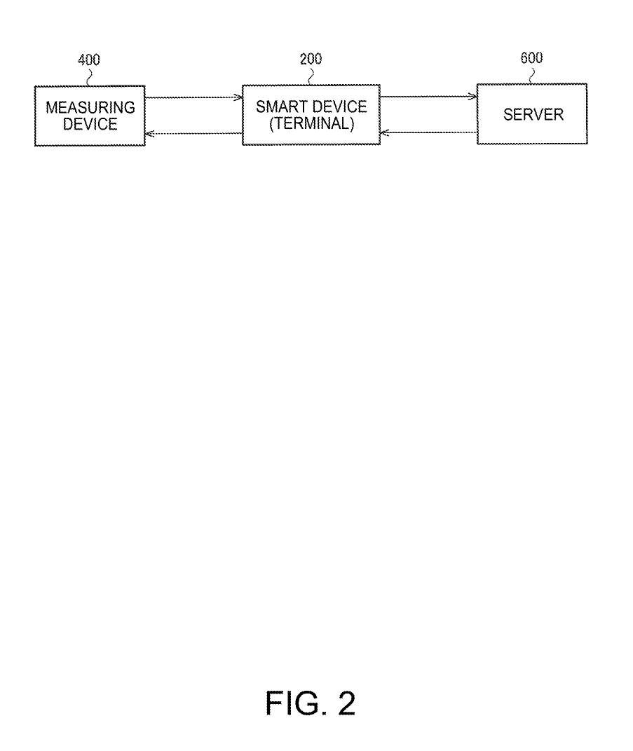

The activity status information computation system 100 may be realized by one measuring device but may also be realized by a measuring device 400, a smart device (terminal) 200 and a server 600 connected together for communication via a network, as shown in FIG. 2. Also, various modifications can be made, such as the activity status information computation system 100 realized by the two devices of the measuring device 400 and the smart device 200.

In the case of FIG. 2, the measuring device 400 has the measuring unit 150. The smart device 200 has the first information acquisition unit 110, the second information acquisition unit 120 and the accepting unit 300. The server 600 has the processing unit 130. Also, the functions of the processing unit 130 may be realized by the two devices of the smart device 200 and the server 600.

### 3. Details of Processing

Next, details of the processing according to the embodiment will be described.

Up to now, the example in which whether to reflect the alteration of the personal biometric setting information or not is decided according to the attribute of the personal biometric setting information is described. In practice, the determination as to whether to reflect the alteration of the personal biometric setting information or not, and at which timing to reflect the alteration when the alteration should be reflected, varies according to information other than the attribute of the personal biometric setting information as well. The information other than the attribute of the personal biometric setting information is, for example, the amount of change in the value of the attribute in the alteration request, the time elapsed since the previous reflection processing of the personal biometric setting information, the time of use of the measuring apparatus for the measured biometric information, and the like.

Therefore, the processing unit 130 carries out the decision processing about the reflection timing or the decision processing about whether to reflect the alteration or not, on the basis of at least one of the amount of change in the value of the attribute in the alteration request, the time elapsed since the previous reflection processing of the personal biometric setting information, and the time of use of the measuring apparatus for the measured biometric information, and on the basis of the attribute.

Thus, even in the case of an alteration of the personal biometric setting information with the same attribute, it is possible discriminate the timing of reflecting the alteration of the setting, and whether to carry out the reflection processing or not, or the like.

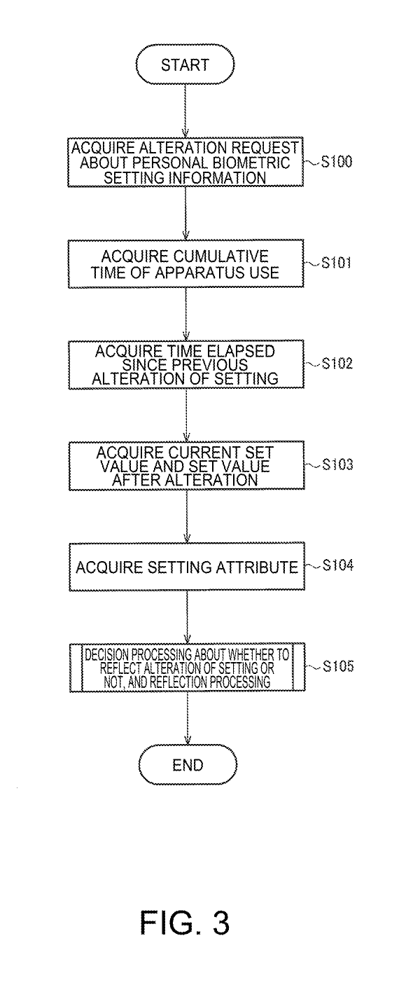

Next, the flow of the processing according to the embodiment will be described, using the flowcharts of FIGS. 3 to 5, 8, 9 and 11. First, an outline of the flow of the overall processing will be described, using the flowchart of FIG. 3.

First, the second information acquisition unit 120 acquires an alteration request about the personal biometric setting information (S100).

Then, the processing unit 130 acquires the cumulative time of apparatus use (time of use) (S101) and subsequently acquires the time elapsed since the timing when the previous alteration of the setting is reflected (S102). Also, the processing unit 130 acquires a current set value of the personal biometric setting information and a set value after the alteration (S103) and acquires the attribute of the personal biometric setting information about which the alteration request is made (S104).

Subsequently, the processing unit 130 carries out decision processing about whether to reflect the alteration of the setting and reflection processing of the alteration of the setting (S105), and then ends the processing. The specific processing contents of the decision processing and the reflection processing carried out in Step S105 vary according to the attribute of the personal biometric setting information to be altered.

Specifically, in Step S105, classification processing as shown in FIG. 4 is carried out before the decision processing and the reflection processing are carried out. First, the processing unit 130 determines whether the attribute of the personal biometric setting information about which the alteration request is made is the weight or not (S201).

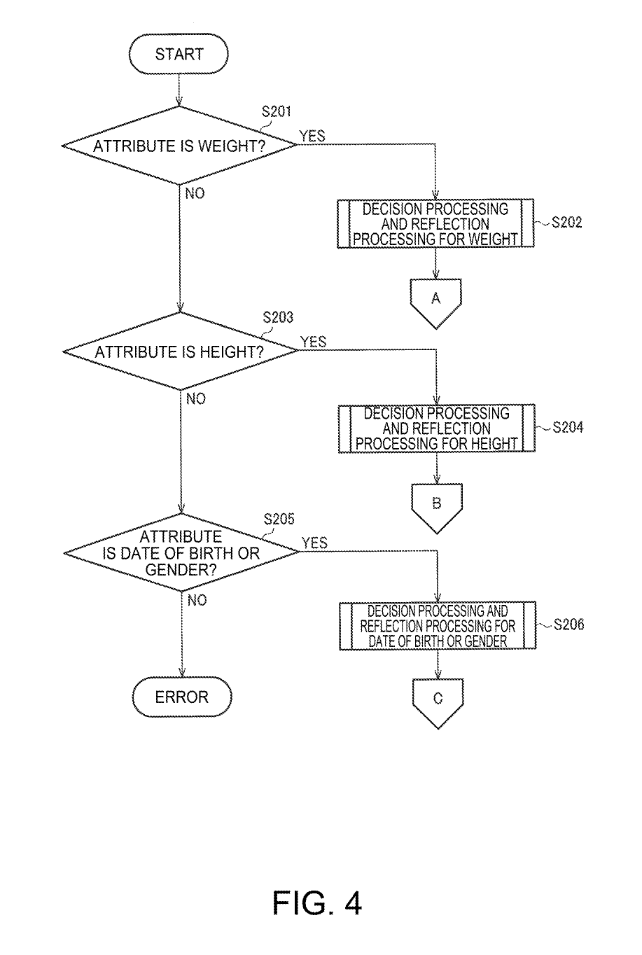

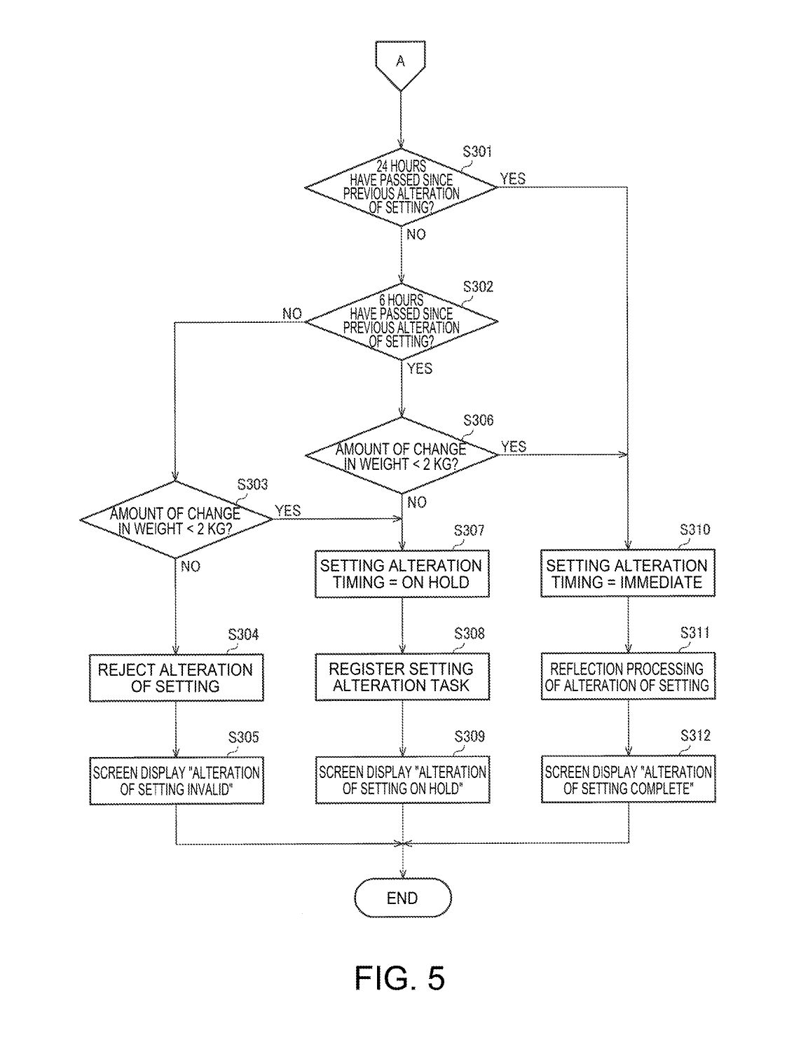

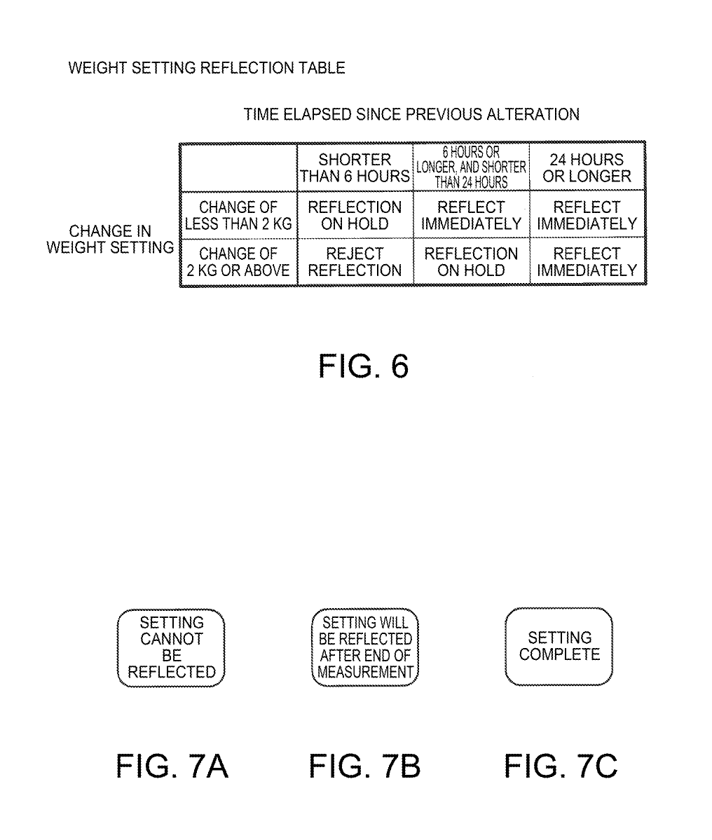

When the processing unit 130 determines that the attribute of the personal biometric setting information about which the alteration request is made is the weight, the processing unit 130 carries out the decision processing or reflection processing of the weight (S202). Details of Step S202 will be described below, using FIGS. 5 and 6.

Meanwhile, when the processing unit 130 determines that the attribute of the personal biometric setting information about which the alteration request is made is not the weight, the processing unit 130 determines whether the attribute is the height or not (S203).

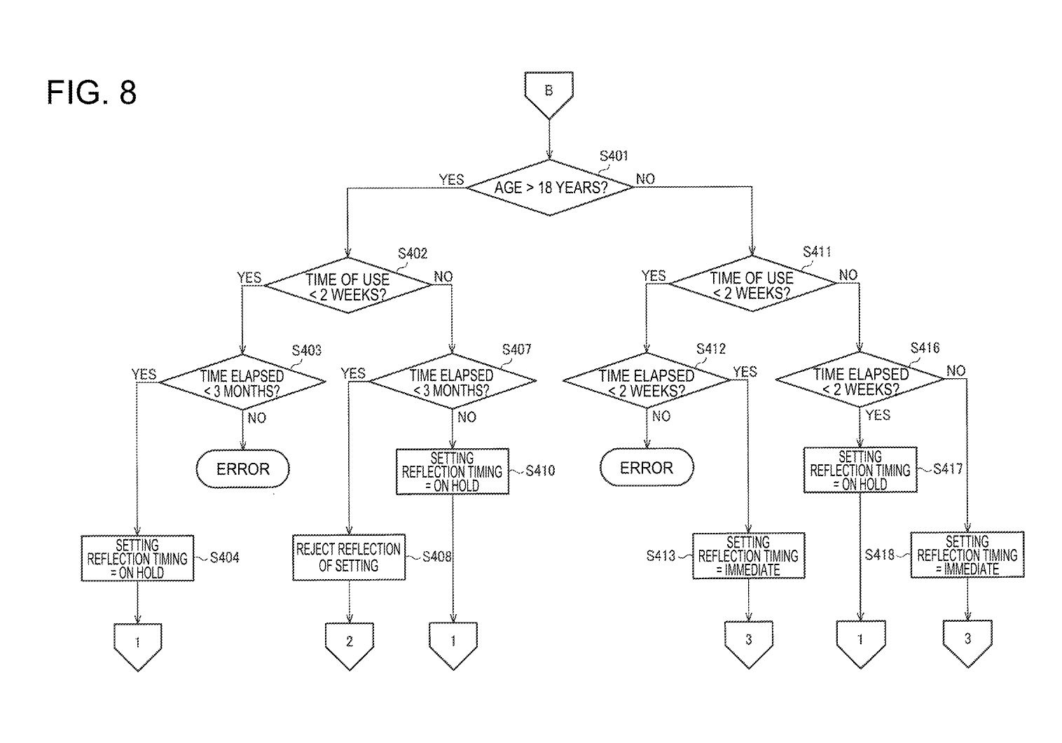

When the processing unit 130 determines that the attribute is the height, the processing unit 130 carries out the decision processing or reflection processing of the height (S204). Details of Step S204 will be described below, using FIGS. 8 to 10B.

Meanwhile, when the processing unit 130 determines that the attribute is not the height, the processing unit 130 determines whether the attribute is the date of birth or gender, or not (S205).

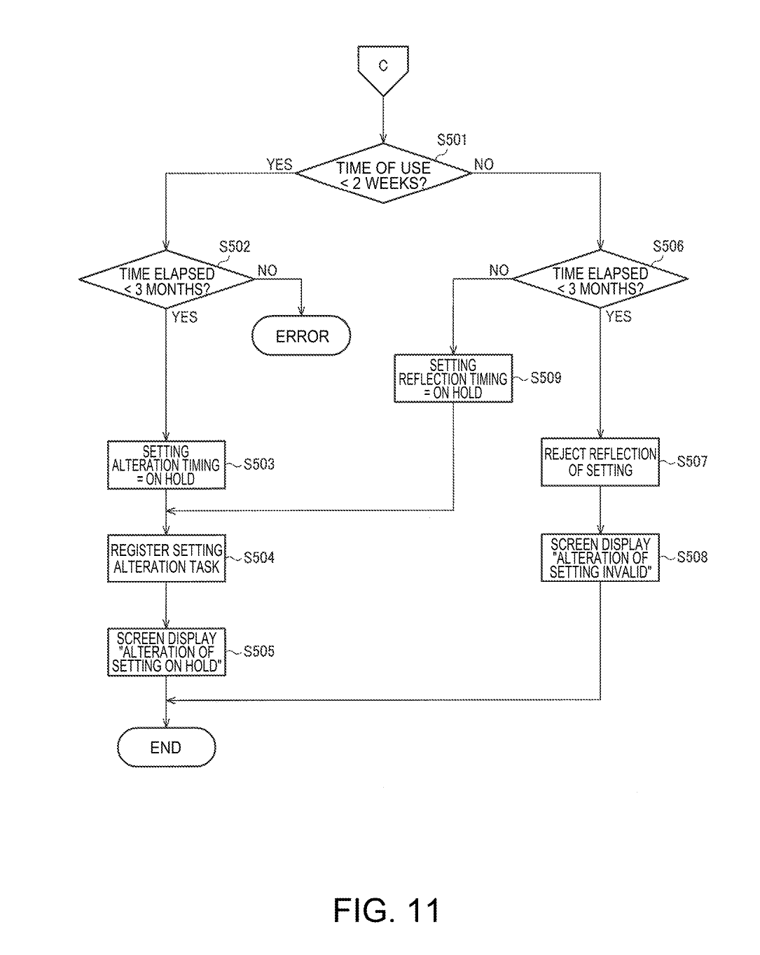

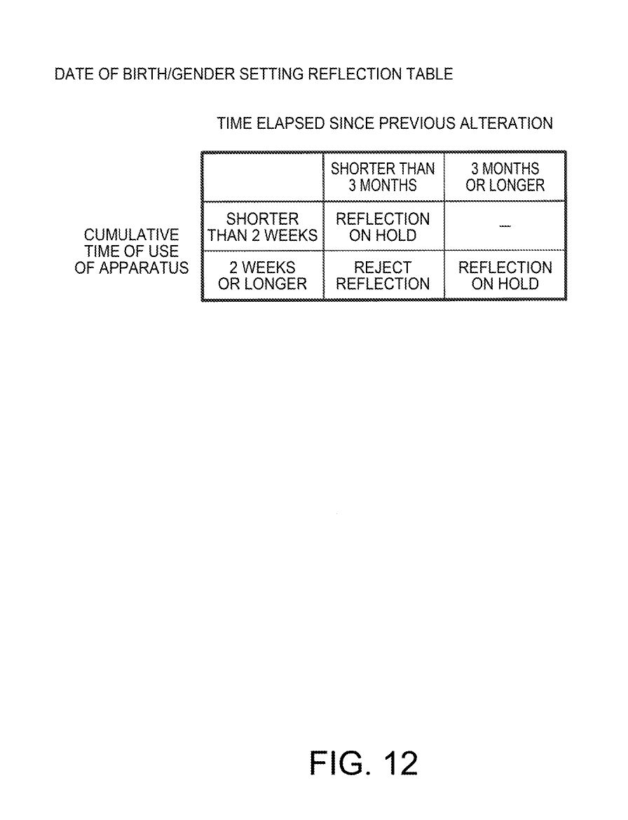

When the processing unit 130 determines that the attribute is the date of birth or gender, the processing unit 130 carries out the decision processing or reflection processing of the date of birth or gender (S206). Details of Step S206 will be described below, using FIGS. 11 and 12.

Meanwhile, when the processing unit 130 determines that the attribute is not the date of birth or gender, the processing unit 130 determines whether this is an error, and ends the processing.

Next, the details of the processing of Step S202 will be described, using the flowchart of FIG. 5.

The processing unit 130 determines whether 24 hours have passed since the timing when the previous alteration of the setting is reflected or not (S301).

When the processing unit 130 determines that 24 hours have not passed since the timing when the previous alteration of the setting is reflected, the processing unit 130 determined whether 6 hours have passed since the timing when the previous alteration of the setting is reflected or not (S302).

When the processing unit 130 determines that 6 hours have not passed since the timing when the previous alteration of the setting is reflected, the processing unit 130 further determines whether the amount of change in the weight is less than 2 kg or not (S303).

When the processing unit 130 determines that the amount of change in the weight is equal to or above 2 kg, the processing unit 130 rejects the alteration of the setting (S304) and shows a screen display indicating that the alteration of the setting is invalid (S305).

That is, as shown in the table of FIG. 6, in the case where the attribute is the weight, the processing unit 130 decides not to carry out the reflection processing of the alteration, that is, rejects the alteration request about the setting, when the time elapsed since the previous reflection processing is shorter than a second time elapsed (6 hours) which is shorter than a first time elapsed (24 hours) and the amount of change is equal to or above a predetermined amount of change (2 kg). In this example, the first time elapsed is 24 hours, the second time elapsed is 6 hours, and the predetermined amount of change is 2 kg. However, the embodiment is not limited these values.

Thus, it is possible not to reflect the alteration of the setting to greatly alter the weight on the computation processing of the activity status information within a short period following the previous reflection timing for the alteration of the setting, or the like. In this case, an image or text as shown in FIG. 7A is shown on a display unit.

Meanwhile, when the processing unit 130 determines in Step S303 that the amount of change in the weight is less than 2 kg, the processing unit 130 decides to put on hold the reflection processing of the alteration of the setting (S307), then registers a task to carryout the alteration of the setting later (S308), and shows a screen display indicating that the alteration of the setting is on hold (S309).

That is, as shown in the table of FIG. 6, in the case where the attribute is the weight, the processing unit 130 carries out the reflection processing of the alteration at a second timing which is later than a first timing corresponding to the alteration request, when the time elapsed since the previous reflection processing is shorter than a second time elapsed (6 hours) which is shorter than a first time elapsed (24 hours) and the amount of change in the attribute value is less than a predetermined amount of change (2 kg).

Thus, it is possible to put on hold the alteration of the setting to slightly alter the weight within a short period following the previous reflection timing for the alteration of the setting, and for example, reflect the alteration of the setting on the computation processing of the activity status information after having the user's confirmation, or the like. In this case, an image or text as shown in FIG. 7B is shown on the display unit.

Also, when the processing unit 130 determines in Step S302 that 6 fours have passed since the timing when the previous alteration of the setting is reflected, the processing unit 130 determines whether the amount of change in the weight is less than 2 kg or not (S306).

Then, when the processing unit 130 determines that the amount of change in the weight is equal to or above 2 kg, the processing unit 130 puts on hold the timing of carrying out the alteration of the setting (second timing) (S307), registers a setting alteration task (S308), and shows a screen display indicating that the alteration of the setting is on hold (S309).

That is, as shown in the table of FIG. 6, in the case where the attribute is the weight, the processing unit 130 carries out the reflection processing of the alteration at the second timing which is later than the first timing corresponding to the alteration of the setting, when the time elapsed is equal to or longer than a second time elapsed (6 hours) and shorter than a first time elapsed (24 hours) and the amount of change is equal to or above a predetermined amount of change (2 kg).

Thus, it is possible to put on hold the alteration of the setting to greatly alter the weight within a predetermined period following the previous reflection timing for the alteration of the setting, and for example reflect the alteration of the setting on the computation processing of the activity status information after having the user's confirmation, or the like. In this case, an image or text as shown in FIG. 7B is shown on the display unit.

Meanwhile, when the processing unit 130 determines that the amount of change in the weight is less than 2 kg, the processing unit 130 sets the timing of the alteration of the setting to immediate (S310), carries out the reflection processing of the alteration of the setting (S311), shows a screen display indicating the completion of the alteration of the setting (S312), and ends the processing.

That is, as shown in the table of FIG. 6, in the case where the attribute is the weight, the processing unit 130 carries out the reflection processing of the alteration at the first timing corresponding to the alteration request, when the time elapsed since the previous reflection processing is equal to or longer than a second time elapsed (6 hours) and shorter than a first time elapsed (24 hours) and the amount of change is less than a predetermined amount of change (2 kg).

Thus, it is possible to immediately reflect the alteration of the setting to slightly alter the weight on the computation processing of the activity status information even during measurement, within a predetermined period following the previous reflection timing for the alteration of the setting, or the like. In this case, an image or text as shown in FIG. 7C is shown on the display unit.

Also, when the processing unit 130 determines in the above Step S301 that 24 hours have passed since the timing when the previous alteration of the setting is reflected, the processing unit 130 sets the timing of the alteration of the setting to immediate (S310), carries out the reflection processing of the alteration of the setting (S311), shows a screen display indicating the completion of the alteration of the setting (S312), and ends the processing.

That is, as shown in the table of FIG. 6, in the case where the attribute is the weight, the processing unit 130 carries out the reflection processing of the alteration at the first timing corresponding to the alteration of the setting, when the time elapsed since the previous reflection processing is equal to or longer than a first time elapsed.

Thus, when a predetermined time or longer has passed since the previous reflection timing of the alteration of the setting, it is possible to immediately reflect the alteration of the weight setting on the computation processing of the activity status information even during measurement, regardless of the amount of change in the weight, or the like. In this case, an image or text as shown in FIG. 7C is shown on the display unit. The above is the flow of the decision processing about whether to reflect an alteration of the weight setting or not and the reflection processing of the alteration of the setting.

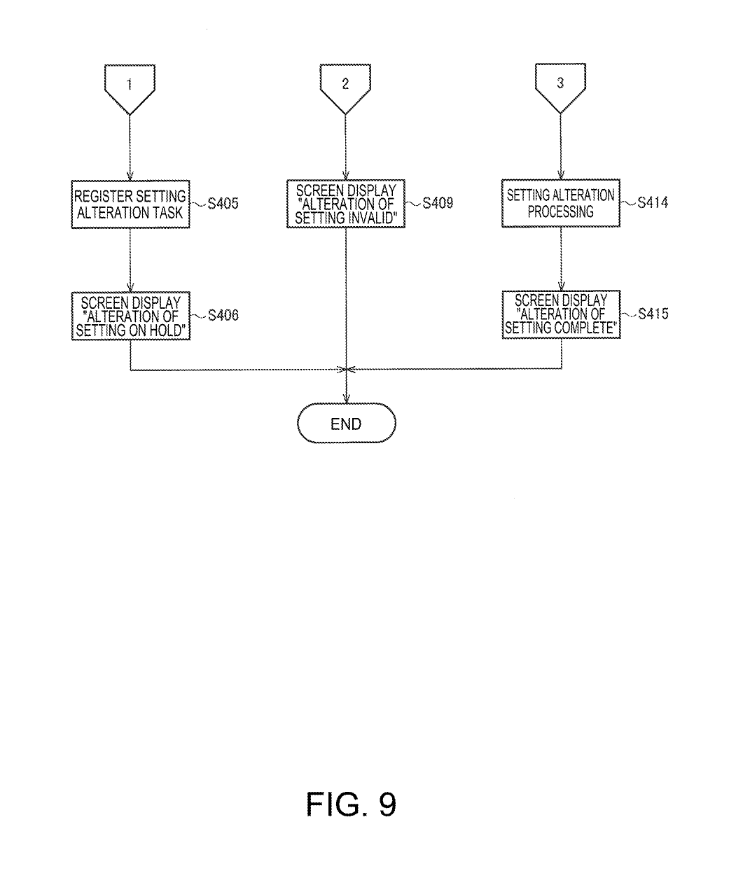

Next, the details of the processing of Step S204 in FIG. 4 will be described, using the flowcharts of FIGS. 8 and 9.

First, the processing unit 130 determines whether the set age of the user is 18 years of age or above, or not (S401). In the embodiment, when the attribute of the persona biometric setting information to be altered is the height of the user, the processing unit 130 carries out the decision processing about the reflection timing or the decision processing about whether to reflect the alteration or not, on the basis of the age or date of birth of the user.

Thus, it is possible to decide whether to reflect the alteration of the setting or not, or to decide the reflection timing when the alteration of the setting is to be reflected, on the basis of different criteria (conditions) depending on the age of the user, or the like.

When it is determined that the set age of the user is 18 years of age or above, whether the time of use of the apparatus (cumulative time of use of the apparatus) is shorter than 2 weeks or not is determined (S402).

When it is determined that the time of use of the apparatus is shorter than 2 weeks, whether the time elapsed since the previous timing when an alteration of the height setting is reflected is shorter than 3 months or not is further determined (S403).

Then, when it is determined that the time elapsed is shorter than 3 months, the timing of carrying out the alteration of the setting is put on hold (second timing) (S404). A setting alteration task is registered (S405) and a screen display indicating that the alteration of the setting is on hold is shown (S406). Then, the processing ends.

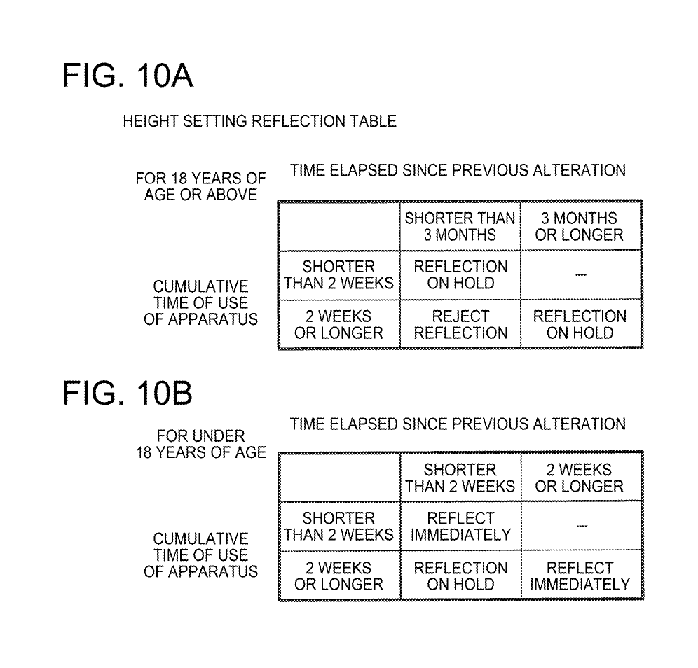

That is, as shown in the table of FIG. 10A, in the case where the attribute is the height of the user, the processing unit 130 carries out the reflection processing of the alteration at a second timing which is later than a first timing (immediate) corresponding to the alteration request, when the age of the user is equal to or above a predetermined threshold (18 years of age), the time of use is shorter than a predetermined time of use (2 weeks), and the time elapsed is shorter than a predetermined time elapsed (3 months). In this example, the predetermined threshold of the age is 18 years of age, the predetermined time of use to be referred to is 2 weeks, and the predetermined time elapsed to be referred to when the user is 18 years old or above is 3 months. However, the embodiment is not limited to these values. These values may be held as table values in a storage unit, not shown.

Thus, for example, when the user is 18 years old or above and the apparatus is in the initial stage of the start of use, and when a predetermined period has not passed since the timing when the previous alteration of the setting is reflected, it is possible to put on hold the alteration of the setting to alter the height, and for example, reflect the alteration of the setting on the computation processing of the activity status information after having the user's confirmation, or the like.

Meanwhile, when it is determined in Step S403 that the time elapsed is 3 months or longer, it is then determined that there is an error, and the processing ends.

Also, when it is determined in Step S402 that the time of use of the apparatus is 2 weeks or longer, whether the time elapsed since the previous timing when an alteration of the height setting is reflected is shorter than 3 months or not is determined (S407). Then, when it is determined that the time elapsed is shorter than 3 months, the alteration of the setting is rejected (S408) and a screen display indicating that the alteration of the setting is invalid is shown (S409). Then, the processing ends.

That is, as shown in the table of FIG. 10A, in the case where the attribute is the height of the user, the processing unit 130 decides not to carry out the reflection processing of the alteration, when the age of the user is equal to or above a predetermined threshold, the time of use is equal to or longer than a predetermined time of use, and the time elapsed is shorter than a predetermined time elapsed.

Thus, for example, when the user is 18 years old or above and the apparatus is not in the initial stage of the start of use, and when only a short period has passed since the timing when the previous alteration of the height setting is reflected, it is possible to reject the alteration of the setting to alter the height, or the like.

Meanwhile, when it is determined in Step S407 that the time elapsed is 3 months or longer, the timing of carrying out the alteration of the setting is put on hold (S410). A setting alteration task is registered (S405) and a screen display indicating that the alteration of the setting is on hold is shown (S406). Then, the processing ends.

That is, as shown in the table of FIG. 10A, in the case where the attribute is the height of the user, the processing unit 130 carries out the reflection processing of the alteration at a second timing which is later than a first timing corresponding to the alteration request, when the age of the user is equal to or above a predetermined threshold, the time of use is equal to or longer than a predetermined time of use, and the time elapsed is equal to or longer than a predetermined time elapsed.

Thus, for example, when the user is 18 years old or above and the apparatus is not in the initial stage of the start of use, and when a predetermined period or longer has passed since the timing when the previous alteration of the height setting is reflected, it is possible to put on hold the alteration of the setting to alter the height, and for example, reflect the alteration of the setting on the computation processing of the activity status information after having the user's confirmation, or the like.

Now, when it is determined in Step S401 that the set age of the user is under 18 years of age, whether the time of use of the apparatus is shorter than 2 weeks or not is determined (S411).

When it is determined that the time of use of the apparatus is shorter than 2 weeks, whether the time elapsed since the previous timing when an alteration of the height setting is reflected is shorter than 2 weeks or not is determined further (S412). When the set age of the user is under 18, the time elapsed to be referred to is 2 weeks.

Then, when it is determined that the time elapsed is shorter than 2 weeks, the timing of the alteration of the setting is set to immediate (S413). The reflection processing of the alteration of the setting is carried out (S414) and a screen display indicating the completion of the alteration of the setting is shown (S415). Then, the processing ends.

That is, as shown in the table of FIG. 10B, in the case where the attribute is the height of the user, the processing unit 130 carries out the reflection processing of the alteration at a first timing corresponding to the alteration request, when the age of the user is below a predetermined threshold, the time of use is shorter than a predetermined time of use, and the time elapsed is shorter than a predetermined time elapsed.

Thus, for example, when the user is under 18 years of age and the apparatus is in the initial stage of the start of use, it is possible to immediately reflect the alteration of the height setting on the computation processing of the activity status information even when a predetermined period has not passed since the timing when the previous alteration of the height setting is reflected, and even during measurement, or the like.

Meanwhile, when it is determined in Step S412 that the time elapsed is 2 weeks or longer, it is then determined that there is an error, and the processing ends.

Also, when it is determined in Step S411 that the time of use of the apparatus is 2 weeks or longer, whether the time elapsed since the previous timing when an alteration of the height setting is reflected is shorter than 2 weeks or not is determined (S416). Then, when it is determined that the time elapsed is shorter than 2 weeks, the timing of carrying out the alteration of the setting is put on hold (second timing) (S417). A setting alteration task is registered (S405) and a screen display indicating that the alteration of the setting is on hold is shown (S406). Then, the processing ends.

That is, as shown in the table of FIG. 10B, in the case where the attribute is the height of the user, the processing unit 130 carries out the reflection processing of the alteration at a second timing which is later than a first timing corresponding to the alteration request, when the age of the user is below a predetermined threshold, the time of use is equal to or longer than a predetermined time of use, and the time elapsed is shorter than a predetermined time elapsed.

Thus, for example, when the user is under 18 years of age and the apparatus is not in the initial stage of the start of use, and when a predetermined period or longer has not passed since the timing when the previous alteration of the height setting is reflected, it is possible to put on hold the alteration of the setting to alter the height, and for example, reflect the alteration of the setting on the computation processing of the activity status information after having the user's confirmation, or the like.

Meanwhile, when it is determined in Step S416 that the time elapsed is 2 weeks or longer, the timing of carrying out the alteration of the setting is set to immediate (S418). The reflection processing of the alteration of the setting is carried out (S414) and a screen display indicating the completion of the alteration of the setting is shown (S415). Then, the processing ends.

That is, as shown in the table of FIG. 10B, in the case where the attribute is the height of the user, the processing unit 130 carries out the reflection processing of the alteration at a first timing corresponding to the alteration request, when the age of the user is below a predetermined threshold, the time of use is equal to or longer than a predetermined time of use, and the time elapsed is equal to or longer than a predetermined time elapsed.

Thus, for example, when the user is under 18 years of age and the apparatus is not in the initial stage of the start of use, and when a predetermined period has passed since the timing when the previous alteration of the height setting is reflected, it is possible to immediately reflect the alteration of the height setting on the computation processing of the activity status information even during measurement, or the like. The above is the flow of the decision processing about whether to reflect an alteration of the height setting and the reflection processing of the alteration of the setting.

Next, the details of Step S206 in FIG. 4 will be described, using the flowchart of FIG. 11.

First, whether the time of use of the apparatus (cumulative time of use of the apparatus) is shorter than 2 weeks or not is determined (S501).

When it is determined that the time of use of the apparatus is shorter than 2 weeks, whether the time elapsed since the previous timing when an alteration of the setting of the date of birth or gender is reflected is shorter than 3 months or not is determined further (S502).

When it is determined that the time elapsed is shorter than 3 months, the timing of carrying out the alteration of the setting is put on hold (S503). A setting alteration task is registered (S504) and a screen display indicating that the alteration of the setting is on hold is shown (S505). Then, the processing ends.

That is, as shown in FIG. 12, in the case where the attribute is the date of birth or gender of the user, the processing unit 130 carries out the reflection processing of the alteration at a second timing that is later than a first timing corresponding to the alteration request, when the time of use is shorter than a predetermined time of use and the time elapsed is shorter than a predetermined time elapsed.

Thus, when the apparatus is in the initial stage of the start of use and a predetermined time has not passed since the timing when the previous alteration of the setting of the date of birth or gender is reflected, it is possible to put on hold the alteration of the setting to alter the date of birth or gender, and for example, reflect the alteration of the setting on the computation processing of the activity status information after having the user's confirmation, or the like.

Meanwhile, when it is determined in Step S502 that the time elapsed is 3 months or longer, it is determined that there is an error, and the processing ends.

Also, when it is determined that the time of use of the apparatus is 2 weeks or longer, whether the time elapsed since the previous timing when an alteration of the setting of the date of birth or gender is reflected is shorter than 3 months or not (S506).

When it is determined that the time elapsed is shorter than 3 months, the alteration of the setting is rejected (S507) and a screen display indicating that the alteration of the setting is invalid is shown (S508). Then, the processing ends.

That is, as shown in FIG. 12, in the case where the attribute is the date of birth of gender of the user, the processing unit 130 decides not to carry out the reflection processing of the alteration when the time of use is equal to or longer than a predetermined time of use and the time elapsed is shorter than a predetermined time elapsed.

Thus, when the apparatus is not in the initial stage of the start of use, and when a predetermined time has not passed since the timing when the previous alteration of the setting of the date of birth or gender is reflected, it is possible to reject the alteration of the setting to alter the date of birth or gender, or the like.

Meanwhile, when it is determined that the time elapsed is 3 months or longer, the timing of carrying out the alteration of the setting is put on hold (S509). A setting alteration task is registered (S504) and a screen display indicating that the alteration of the setting is on hold is shown (S505). Then, the processing ends.

That is, as shown in FIG. 12, in the case where the attribute is the date of birth of gender of the user, the processing unit 130 carries out the reflection processing of the alteration at a second timing that is later than a first timing corresponding to the alteration request, when the time of use is equal to or longer than a predetermined time of use and the time elapsed is equal to or longer than a predetermined time elapsed.

Thus, when the apparatus is not in the initial stage of the start of use, and when a predetermined time has passed since the timing when the previous alteration of the setting of the date of birth or gender is reflected, it is possible to put on hold the alteration of the setting to alter the date of birth or gender, and for example, reflect the alteration of the setting on the computation processing of the activity status information after having the user's confirmation, or the like.

### 4. Example of Configuration of Measuring Device

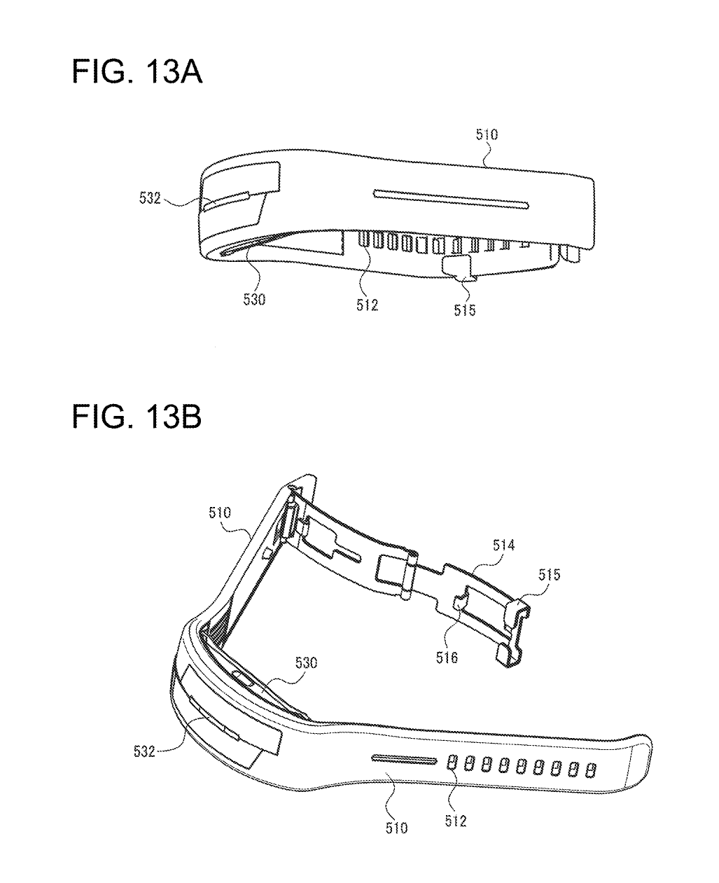

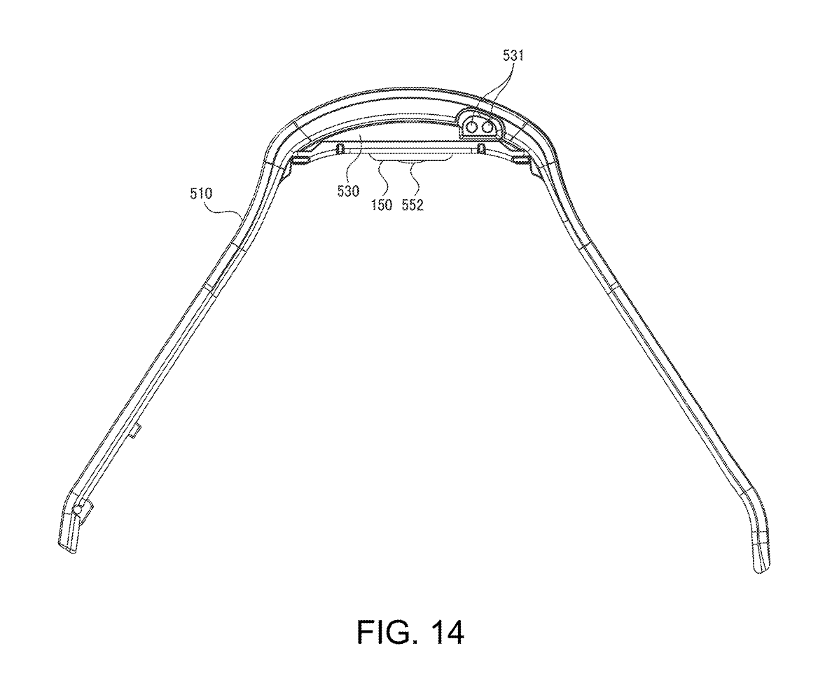

FIGS. 13A, 13B and 14 show the external appearance of the measuring device 400 (biometric information detection device) according to the embodiment. FIG. 13A shows the measuring device 400, as viewed from the front. FIG. 13B is a view from above. FIG. 14 is a view from the side.

As shown in FIGS. 13A to 14, the measuring device 400 has a band portion 510, a case portion 530, and a sensor unit (measuring unit) 150. The case portion 530 is attached to the band portion 510. The sensor unit 150 is provided on the case portion 530. However, the measuring device 400 of this embodiment is not limited to the configurations shown in FIGS. 13A to 14 or the like and various modifications can be made such as omitting a part of the components, replacing a part of the components with another component, and adding another component.

The band portion 510 is to be wound on the user's wrist so that the user can wear the measuring device 400. The band portion 510 has a hole part 512 and a buckle part 514. The buckle part 514 has a band insertion part 515 and a protrusion 516. The user can wear the measuring device 400 on the wrist by inserting one end side of the band portion 510 into the band insertion part 515 of the buckle part 514 and inserting the protrusion 516 of the buckle part 514 into the hole part 512 of the band portion 510. In this case, the magnitude of pressure on the sensor unit 150 (pressure on the wrist surface) is adjusted according to which hole part 512 the protrusion 516 is inserted in.

The case portion 530 is equivalent to the main body part of the measuring device 400. Inside the case portion 530, various components of the measuring device 400 such as sensor unit 150, the first information acquisition unit 110, the second information acquisition unit 120 and the processing unit 130 are provided. That is, the case portion 530 is a casing which accommodates these components.

A light emitting window part 532 is provided on the case portion 530. The light emitting window part 532 is made of a light-transmitting member. A light emitting unit (LED) mounted on a flexible board is provided in the case portion 530, and the light from this light emitting unit is cast outside the case portion 530 via the light emitting window part 532.

As shown in FIG. 14, a terminal part 531 is provided on the case portion 530. As the measuring device 400 is installed on a cradle, not shown, the terminal part of the cradle and the terminal part 531 on the case portion 530 are electrically connected to each other. This enables charging of a rechargeable battery provided in the case portion 530.

The sensor unit 150 is to detect measured biometric information such as the pulse waves of a subject. For example, the sensor unit 150 has a light receiving part and a light emitting part. Also, the sensor unit 150 has a protruding part 552 which is made of a light-transmitting member and which contacts and pressurizes the skin surface of the subject. In the state where the protruding part 552 is thus pressurizing the skin surface, the light emitting part emits light and the light receiving part receives the light reflected from the subject (blood vessel). The result of the light reception is outputted as a detection signal to the first information acquisition unit 110. The measured biometric information to be detected by the measuring device 400 of the embodiment is not limited to the pulse waves (pulse rate). The measuring device 400 may be a device which detects measured biometric information other than pulse waves (for example, blood oxygen saturation level, body temperature, heart rate, and the like).

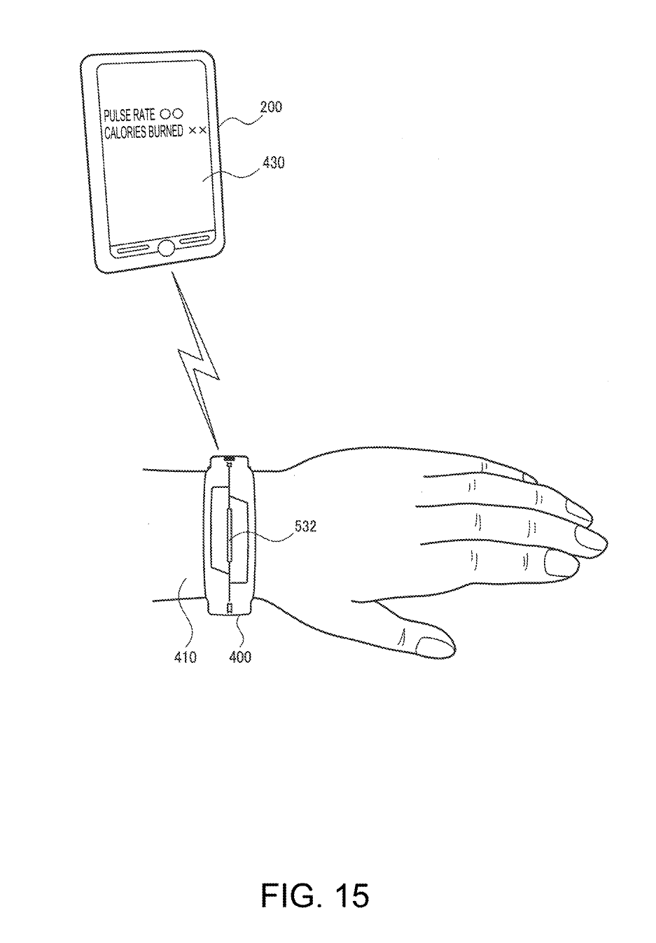

FIG. 15 is an explanatory view about the installation of the measuring device 400 and the communication thereof with an information processing device 200.

As shown in FIG. 15, the user as a subject installs the measuring device 400 on a wrist 410, similarly to a wristwatch. The sensor unit 150 is provided on the surface of the case portion 530 on the side of the subject, as shown in FIG. 14. Therefore, as the measuring device 400 is installed, the protruding part 552 of the sensor unit 150 contacts and pressurizes the skin surface of the wrist 410. In this state, the light emitting part of the sensor unit 150 emits light and the light receiving part receives the reflected light. Thus, measured biometric information such as pulse waves is detected.

The measuring device 400 and the information processing device 200 are connected for communication and are able to exchange data. The information processing device 200 is a mobile terminal device, for example, a smartphone, mobile phone, feature phone or the like. Alternatively, the information processing device 200 may be an information processing terminal such as a tablet computer. For the communication connection between the measuring device 400 and the information processing device 200, for example, near field radio communication based on Bluetooth or the like can be employed. As the measuring device 400 and the mobile terminal device 200 are thus connected for communication, various kinds of information (measured biometric information, activity status information) such as pulse rate and calories burned can be displayed on a display unit 430 (LCD or the like) of the mobile terminal device 200. That is, various kinds of information found on the basis of the detection signal from the sensor unit 150 can be displayed. The computation processing of the information such as pulse rate and calories burned may be executed in the measuring device 400, or at least a part of the computation processing may be executed in the mobile terminal device 200.

In the activity status information computation system or the like according to the embodiment, a part or a majority of the processing may be realized by a program. In this case, the activity status information computation system or the like of the embodiment is realized as a processor such as a CPU executes the program. Specifically, the program stored in a non-temporary information storage device is read out and the program thus read out is executed by the processor such as a CPU. Here, the information storage device (computer-readable device) is for storing a program and data, and the functions thereof can be realized by an optical disk (DVD, CD or the like), HDD (hard disk drive), or memory (card memory, ROM or the like), or the like. The processor such as a CPU then carries out the various kinds of processing according to the embodiment, on the basis of the program (data) stored in the information storage device. That is, a program for causing a computer (device having an operation unit, a processing unit, a storage unit and an output unit) to function as each part in the embodiment (program for causing the computer to execute the processing of each part) is stored in the information storage device.

The activity status information computation system or the like of the embodiment may also include a processor and a memory. The processor in this case may be, for example, a CPU (central processing unit). However, the processor is not limited to the CPU, and various processors such as a GPU (graphics processing unit) or DSP (digital signal processor) can be used. The processor may also be a hardware circuit based on an ASIC (application specific integrated circuit). The memory is for storing a computer-readable command. Each part of the activity status information computation system or the like of the embodiment is realized as the command is executed by the processor. The memory in this case may be a semiconductor memory such as an SRAM (static random access memory) or DRAM (dynamic random access memory), or may be a register, hard disk or the like. The command in this case may be a command of a command set forming a program, or may be a command to instruct the hardware circuit of the processor to carry out an operation.

The embodiment is described in detail above. However, a person skilled in the art can readily understand that a number of modifications can be made without substantially departing from the new matter of the invention and its effects. Therefore, such modifications should be included in the scope of the invention. For example, a term described at least once with a different term with a broader or same meaning in the specification or drawings can be replaced with the different term at any point in the specification or drawings. Also, the configurations and operations of the activity status information computation system are not limited to those described in the embodiment and various modifications can be made.

What is claimed is:

1. An activity status information computation system comprising:
a first information acquisition unit which acquires measured biometric information measured about a user;
a second information acquisition unit which acquires personal biometric setting information set as personal information of the user; and
a processing unit which carries out computation processing of activity status information of the user on the basis of the measured biometric information and the personal biometric setting information;
wherein when there is an alteration request about the personal biometric setting information, the processing unit carries out decision processing about a timing of reflecting the alteration of the personal biometric setting information on the computation processing of the activity status information, or decision processing about whether to reflect the alteration or not, according to an attribute of the personal biometric setting information.

2. The activity status information computation system according to claim 1, wherein
when the attribute is a first attribute, the processing unit carries out reflection processing of the alteration at a first timing corresponding to the alteration request, and
when the attribute is a second attribute, the processing unit does not carry out the reflection processing or carries out the reflection processing at a second timing that is later than the first timing.

3. The activity status information computation system according to claim 1, wherein
the processing unit carries out the decision processing about the reflection timing or the decision processing about whether to reflect the alteration or not, on the basis of at least one of an amount of change in value of the attribute in the alteration request, time elapsed since the previous reflection processing of the personal biometric setting information, and time of use of a measuring apparatus for the measured biometric setting information, and on the basis of the attribute.

4. The activity status information computation system according to claim 3, wherein
the attribute is the weight of the user, and
the processing carries out the reflection processing of the alteration at a first timing corresponding to the alteration request when the time elapsed since the previous reflection processing is equal to or longer than a first time elapsed.

5. The activity status information computation system according to claim 3, wherein
the attribute is the weight of the user, and
the processing unit decides not to carry out the reflection processing of the alteration when the time elapsed since the previous reflection processing is shorter than a second time elapsed which is shorter than the first time elapsed and the amount of change is equal to or greater than a predetermined amount of change.

6. The activity status information computation system according to claim 3, wherein
the attribute is the weight of the user, and
the processing unit carries out the reflection processing of the alteration at a second timing that is later than a first timing corresponding to the alteration request, when the time elapsed is equal to or longer than a second time elapsed and shorter than a first time elapsed and the amount of change is equal to or greater than a predetermined amount of change.

7. The activity status information computation system according to claim 3, wherein
the attribute is the weight of the user, and
the processing unit carries out the reflection processing of the alteration at a first timing corresponding to the alteration request, when the time elapsed since the previous reflection processing is equal to or longer than a second time elapsed and shorter than a first time elapsed and the amount of change in the value of the attribute is less than a predetermined amount of change.

8. The activity status information computation system according to claim 3, wherein
the attribute is the weight of the user, and
the processing unit carries out the reflection processing of the alteration at a second timing that is later than a first timing corresponding to the alteration request, when the time elapsed since the previous reflection processing is shorter than a second time elapsed which is shorter than a first time elapsed and the amount of change in the value of the attribute is less than a predetermined amount of change.

9. The activity status information computation system according to claim 3, wherein
the attribute is the height of the user, and
the processing unit carries out the decision processing about the reflection timing or the decision processing about whether to reflect the alteration or not, on the basis of the age or date of birth of the user.

10. The activity status information computation system according to claim 3, wherein
the attribute is the height of the user, and
the processing unit carries out the reflection processing of the alteration at a first timing corresponding to the alteration request, when the age of the user is below a predetermined threshold, the time of use is shorter than a predetermined time of use, and the time elapsed is shorter than a predetermined time elapsed.

11. The activity status information computation system according to claim 3, wherein
the attribute is the height of the user, and
the processing unit carries out the reflection processing of the alteration at a second timing that is later than a first timing corresponding to the alteration request, when the age of the user is below a predetermined threshold, the time of use is equal to or longer than a predetermined time of use, and the time elapsed is shorter than a predetermined time elapsed.

12. The activity status information computation system according to claim 3, wherein
the attribute is the height of the user, and
the processing unit carries out the reflection processing of the alteration at a first timing corresponding to the alteration request, when the age of the user is below a predetermined threshold, the time of use is equal to or longer than a predetermined time of use, and the time elapsed is equal to or longer than a predetermined time elapsed.

13. The activity status information computation system according to claim 3, wherein
the attribute is the height of the user, and
the processing unit carries out the reflection processing of the alteration at a second timing that is later than a first timing corresponding to the alteration request, when the age of the user is equal to or above a predetermined threshold, the time of use is shorter than a predetermined time of use, and the time elapsed is shorter than a predetermined time elapsed.

14. The activity status information computation system according to claim 3, wherein
the attribute is the height of the user, and
the processing unit decides not to carry out the reflection processing of the alteration when the age of the user is equal to or above a predetermined threshold, the time of use is equal to or longer than a predetermined time of use, and the time elapsed is shorter than a predetermined time elapsed.

15. The activity status information computation system according to claim 3, wherein
the attribute is the height of the user, and
the processing unit carries out the reflection processing of the alteration at a second timing that is later than a first timing corresponding to the alteration request, when the age of the user is equal to or above a predetermined threshold, the time of use is equal to or longer than a predetermined time of use, and the time elapsed is equal to or longer than a predetermined time elapsed.

16. The activity status information computation system according to claim 3, wherein
the attribute is the date of birth or gender of the user, and
the processing unit carries out the reflection processing of the alteration at a second timing that is later than a first timing corresponding to the alteration request, when the time of use is equal to or longer than a predetermined time of use and the time elapsed is equal to or longer than a predetermined time elapsed.

17. The activity status information computation system according to claim 3, wherein
the attribute is the date of birth or gender of the user, and
the processing unit decides not to carry out the reflection processing of the alteration when the time of use is equal to or longer than a predetermined time of use and the time elapsed is shorter than a predetermined time elapsed.

18. The activity status information computation system according to claim 3, wherein
the attribute is the date of birth or gender of the user, and
the processing unit carries out the reflection processing of the alteration at a second timing that is later than a first timing corresponding to the alteration request, when the time of use is shorter than a predetermined time of use and the time elapsed is shorter than a predetermined time elapsed.
